(19) United States
(12) Patent Application Publication
(10) Pub. No.: US 2024/0124826 A1
(43) Pub. Date: Apr. 18, 2024

(54) ELECTROMAGNETICALLY DRIVEN UNIAXIAL CELL STRETCHING DEVICE

(71) Applicant: UNIVERSITY OF NORTH TEXAS, Dallas, TX (US)

(72) Inventors: Yong YANG, Dallas, TX (US); Michelle Leyi YANG, Dallas, TX (US); Catalina ZUO, Dallas, TX (US); Zhenyu ZUO, Dallas, TX (US)

(21) Appl. No.: 18/486,887

(22) Filed: Oct. 13, 2023

Related U.S. Application Data
(60) Provisional application No. 63/379,585, filed on Oct. 14, 2022.

(51) Int. Cl. C12M 1/42; C12M 1/00; C12M 3/00

(52) U.S. Cl. CPC C12M 35/04; C12M 23/26; C12M 23/46; C12M 35/02

(57) ABSTRACT

An electromagnetically driven one-dimensional stretching device includes a stretchable surface, and an electromagnetic actuator coupled to the stretchable surface. The electromagnetic actuator is configured to stretch the stretchable surface along one-dimension. A method can include spin-coating a weight ratio of about 10:1 about 1:1 of a polydimethylsiloxane resin to a curing agent mixture on a surface to form a stretchable surface, removing the stretchable surface from the surface, coupling an electromagnetic actuator to the stretchable surface, and stretching the stretchable surface along one-dimension using the electromagnetic actuator.

### CROSS-REFERENCE TO RELATED APPLICATIONS

This application claims the benefit of U.S. Provisional Application No. 63/379,585, filed on Oct. 14, 2022, and entitled “ELECTROMAGNETICALLY DRIVEN UNIAXIAL CELL STRETCHING DEVICE,” which is incorporated herein by reference in its entirety for all purposes.

### STATEMENT REGARDING FEDERALLY FUNDED RESEARCH OR DEVELOPMENT

None.

### BACKGROUND

Cardiovascular diseases causes an estimated 17.9 million deaths globally each year (World Health Organization). Endothelial cells that line the vasculature and the endocardium can be subjected to cyclic mechanical stretch. Deviation from physiological stretch may alter the endothelial function, having the risk of atherosclerosis and myocardial infarction. Cell culture platforms have been developed that provide mechanical stretch. However, most of these platforms have fixed strain and frequency, sometimes not in the pathophysiological range.

As a result, most of these existing platforms are hindered by a fixed strain and frequency, non-uniform strain, or require direct physical contact with the cells, all unfit for the observation of cell behavior in the pathophysiological range. In addition, the commercially available Flexcell Systems cost tens of thousand dollars. Therefore, there is a need for a simple, cost-effective, and versatile stretching device for studying the role of pathophysiologically relevant mechanical stretch in cell regulation.

### SUMMARY

In some embodiments, an electromagnetically driven one-dimensional stretching device comprises a stretchable surface, and an electromagnetic actuator coupled to the stretchable surface. The electromagnetic actuator is configured to stretch the stretchable surface along one-dimension.

In some embodiments, a method comprises spin-coating a weight ratio of about 10:1 about 1:1 of a polydimethylsiloxane resin to a curing agent mixture on a surface to form a stretchable surface, removing the stretchable surface from the surface, coupling an electromagnetic actuator to the stretchable surface, and stretching the stretchable surface along one-dimension using the electromagnetic actuator.

In some embodiments, a method of forming strain gradient stretchable surface comprises spin-coating a weight ratio of about 20:1 to about 1:1 of a polydimethylsiloxane resin to a curing agent mixture on a plastic film. The polydimethylsiloxane resin and the curing agent mixture are spin-coated on one end touching the plastic film and another end by two glass members to form a thickness gradient.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1A is a schematic exploded view of an embodiment of an electromagnetic stretching device.

FIG. 1B is a perspective view of an embodiment of the electromagnetic stretching device.

FIG. 1C is a perspective, pictorial view of an embodiment of the electromagnetic stretching device.

FIG. 2A is a schematic perspective view of an embodiment of a uniaxial cell stretching part.

FIG. 2B is a schematic cross-sectional view of an embodiment of the uniaxial cell stretching part.

FIG. 2C is a perspective, pictorial view of an embodiment of the uniaxial cell stretching part.

FIG. 3 is a top, plan, and pictorial view of an embodiment of an electromagnetic actuator.

FIG. 4A is a schematic depiction of an embodiment of a SolidWorks® simulation of the strain gradient of the dimensions of the tapered membrane.

FIG. 4B is a top view of an embodiment of a SolidWorks® simulation of the strain gradient of the dimensions of the tapered membrane.

FIG. 4C is a cross-sectional view of an embodiment of a SolidWorks® strain simulation of the strain gradient of the dimensions of the tapered membrane.

FIG. 5 is an optical view of an embodiment of marked dots on the membrane before and after exposed to 15% overall strain.

FIG. 6A depicts immunofluorescence images of hCMECs subjected to mechanical stretch of various strains in comparison with the static condition of an embodiment of a mechanical strain-dependent cell/nuclear orientation.

FIG. 6B are graphical depictions of an embodiment of quantitative analysis of nuclear orientation of hCMECs under the static condition and various strains.

FIG. 6C is a schematic depiction of an embodiment of an illustrative definition of the nuclear orientation angle θ.

FIG. 7A is a graphical depiction of an embodiment of dot plots of nuclear elongation of hCMECs under static condition and various mechanical strains.

FIG. 7B is a graphical depiction of an embodiment of a comparison in nuclear orientation under static condition and various strains.

FIG. 8A is a schematic depiction of a simulation of fluorescence labeling and recognition of a fluorescence micropillar-labeled PDMS membrane in the unstrained state and at an overall strain of 15%.

FIG. 8B is a schematic depiction of a simulation of fluorescence labeling and recognition of a computer recognition of a circle and an ellipse by removing the original fluorescence marker.

FIG. 8C is a schematic depiction of a simulation of fluorescence labeling and recognition of a strain rate and a direction depicted by arrows.

FIG. 9A is a schematic depiction of an embodiment of a SolidWorks® simulation of measured top strain rates of the PDMS membrane.

FIG. 9B is a schematic depiction of an embodiment of a SolidWorks® simulation of measured bottom strain rates of the PDMS membrane.

### DETAILED DESCRIPTION

As described herein, a multiple of an element, e.g., a clamp or a dowel, may be used, such as, respectively, two clamps and eight dowels, with only a single element, e.g., a clamp or a dowel, of the multiple numbered in the drawing figures to reduce clutter.

Mechanical stretch can play a role in the development, homeostasis, and pathogenesis of human tissues and organs. For example, smooth muscle cells experience one-dimensional (1-D) stretching when an arm moves via muscle contraction, and endothelial cells lining the vascular system undergo physiological cyclic mechanical stretch due to pulsatile blood pressure and circulating blood flow through arteries, veins, and capillaries. On the other hand, abnormal stretching processes are often associated with organ dysfunction and disease. For example, deviation from physiological mechanical stretch can alter the endothelial cell phenotype and function, which can lead to pathological changes and result in cardiovascular diseases such as atherosclerosis (buildup of plaque in arteries) and myocardial infarction (heart attack). Abnormally low esophageal traction forces can result in difficulty in swallowing, or functional dysphagia. It is thus desirable to understand the role of mechanical stretch in the normal function of cells, tissue, and organs, and human disease progression.

To address this issue, cell culture platforms have been developed to provide mechanical stretches. These devices have utilized different mechanisms of stretching a flexible membrane, including mechanical actuation (via a motor), pneumatic actuation (via pressure with fluids), piezoelectric actuation (via electrical energy to generate mechanical displacement), and optical actuation (via energy and light). An electromagnetically driven, uniaxial stretching device, where cells can be grown on a flexible membrane (e.g., a polydimethylsiloxane (PDMS) membrane) mounted onto a 3-D printed track would be desirable.

Disclosed herein is an electromagnetically driven one-dimensional (1-D) stretching device that can provide a simple, versatile, cost-effective, and pathophysiologically relevant platform that can enable the study of the effects of mechanical strain, both normal and abnormal, on cells and tissues. The ability for this device to span the pathophysiological range can allow the observation of the behavior of cells under normal and hypertension conditions and understand its effects on targeted cells, tissues, and organs contributing to various diseases. This device can also provide a useful in vitro platform for other biological studies involving mechanical stretching.

The strain of the membrane can be readily controlled by tailoring the track design and the frequency can be determined by electromagnetic actuation. Furthermore, the mechanical strain gradient can be generated on a membrane with a tapered thickness. This strain gradient, ranging from about 1.5% to about 40%, covers both physiological and pathological vascular stretch ranges. In some embodiments, when human vascular endothelial cells are subjected to the cyclic stretch, the cells can exhibit strain-dependent cell and nuclear orientation and elongation perpendicular to the stretching direction, compared to the random cell and nuclear orientation under the static condition. However, the overstretching can lead to deviation from the aforementioned orientation and elongation, and impaired the tight junctions, leading to a leaky endothelium. The disclosed versatile, cost-effective, pathophysiologically relevant stretching device can provide a useful platform for advancement of vascular disease research and treatment.

In some embodiments, the device applies an electromagnetic actuator to generate uniaxial stretch of pathophysiological relevance on a flexible membrane where cells are grown. Except for the electromagnetic actuator, the device can encompass a stretching part (moving component, fixed component, a stretchable surface such as a membrane, and track) and a container. In some aspects, all of the components can be 3-D printed at low cost (less than $100 compared with about $50,000 for a commercial device) and can be readily tailored for various applications (with uniform mechanical strain over large areas and can fit in all pathophysiological ranges).

In some embodiments, the device solves four problems including: 1) forming a defined, uniform strain or strain gradient over large areas, 2) providing a low cost solution, 3) providing a versatile strain and frequency to fit in pathophysiological ranges, and 4) eliminating potential contamination in cell culture. Mechanical stretch may play a role in the human body with its importance to the function and homeostasis of cells, tissues, and organs. For example, endothelial cells lining the vascular system may undergo physiological cyclic mechanical deformation due to pulsatile blood pressure and circulating blood flow through arteries, veins, and capillaries. Deviation from physiological mechanical stretch can alter the endothelial cell phenotype and function, which can lead to pathological changes and result in cardiovascular diseases, such as atherosclerosis (buildup of plaque in arteries) and myocardial infarction (heart attack). Of note, the dysfunction of human cerebral microvascular endothelial cells (hCMECs) can contribute to the onset and progression of neurodegenerative diseases such as Alzheimer's and Parkinson's diseases. The physiological range of vascular mechanical strain in vivo can be 5-10% and a strain above 20% is often seen in hypertension, leading to serious, often fatal health conditions. In the cardiovascular system, hypertension forces the heart to work harder to pump blood throughout the body due to the thickening and hardening of arteries. To understand the role of mechanical stretch in the normal function of cells, tissue, and organs, and human disease progression can be beneficial.

Cell culture platforms that provide mechanical stretch, from a 1-D uniaxial stretch, a two-dimensional (2-D) biaxial or circumferential stretch, to a three-dimensional (3-D) radial stretch, have been developed. These devices have utilized different mechanisms of stretching a flexible membrane, as described herein. However, many of these existing platforms are hindered by a fixed strain and frequency, non-uniform strain, or require direct physical contact with the cells, all of which are generally unfit for the observation of cell behavior in the pathophysiological range. For example, an exemplary reported electromagnetically actuated device usually has multiple downfalls including a non-physiologically relevant magnitude and frequency of strain as well as non-uniform strain. Therefore, there is generally a need for pathophysiologically relevant, effective in vitro models to study the role of mechanical stretch in cell regulation.

A simple, cost-effective, electromagnetically driven 1-D stretching device with versatile strain, frequency, and potentially material properties is desirable, particularly by generating a strain gradient using a tapered, stretchable membrane. The formation of strain gradients of pathophysiological relevance can be confirmed by computational and experimental characterization of the stretchable membrane. Furthermore, vascular endothelial cells, using hCMECs as model cells, grown on the stretchable membrane in the device can respond differently to the various strains along the gradient.

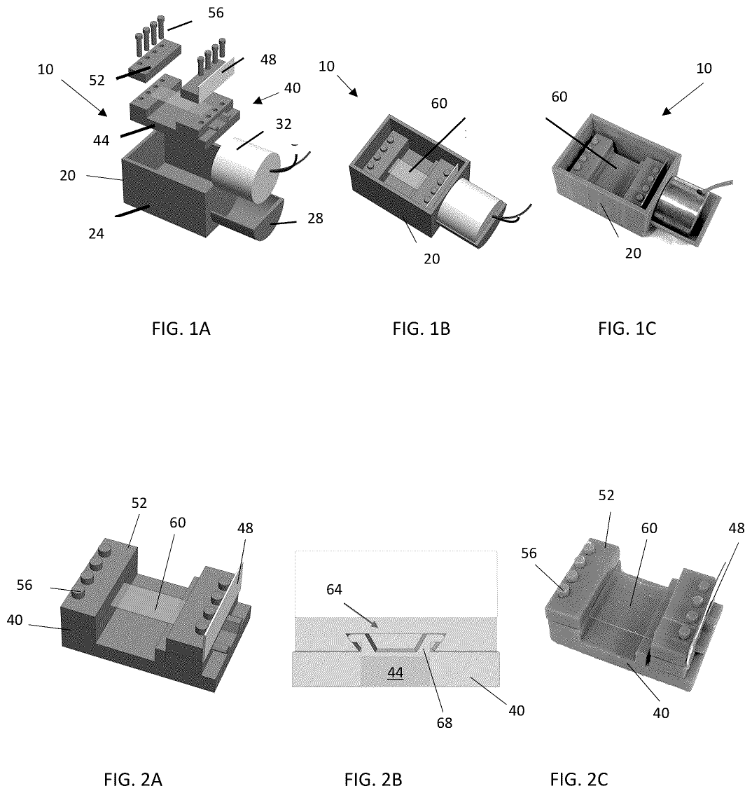

Referring to FIGS. 1A-1C, in some embodiments, an electromagnetic stretching device 10 or stretching device 10 can include or consist of a stretching part 20 and an electromagnetic actuator 70. FIGS. 1A-1C depict a design and photo of the (A) an exploded view, (B) an assembled view, and (C) a photo of the device 10. The stretching part 20 may comprise several components, including a uniaxial cell stretching assembly 40 having a track 44, a metal piece 48, at least one clamp 52, such as two clamps 52, and multiple dowels 56, and in the depicted embodiment eight dowels 56, and a container 24 forming a magnet holder 28 containing an electromagnet 32. In some embodiments, adhesives may be used instead of dowels 56 to bond the stretchable surface 60 such as a flexible membrane on the clamps 52. All these components can be designed (e.g., using design software such as SolidWorks® by Dassault Systems SOLIDWORKS Co., Waltham, MA, USA) and printed with a 3-D printer (e.g., Elegoo Standard photopolymer resin (Elegoo, Shenzhen, China) using a 3-D printer from Form 3, Formlabs, Somerville, MA, USA). In some embodiments, the electromagnetic stretching device 10 can apply the electromagnetic actuator 70 to generate uniaxial stretch on a stretchable surface 60 where cells are grown. Except for the electromagnetic actuator 70, the electromagnetic stretching device 10 can encompass the stretching part 20 (a moving component 64, a fixed component 68, the stretchable surface 60, and the track 44 as depicted in FIGS. 2A-2C) and a container 24, all being optionally 3-D printed at low cost (about $30 USD) and can be readily tailored for various applications (with uniform mechanical strain over large areas and can fit in all pathophysiological ranges).

The stretchable surface or stretchable membrane 60 can be made from any suitable membrane such as a polymeric membrane. In some aspects, the stretchable membrane 60 can be made of PDMS (e.g., as available from Sylgard 184, Dow Corning, Midland, MI, USA). In some aspects, the stretchable surface 60 can be prepared by spin-coating a mixture of PDMS resin and curing agent at a weight ratio of about 20:1 to about 1:1, about 15:1 to about 1:1, about 10:1 to about 1:1, or about 10:1 to about 1.05:1, on a plastic film, which can be pre-trimmed into a rectangle, at about 100 revolutions-per-minute (rpm), about 200 rpm, about 300 rpm, or about 400 rpm, or about 300 rpm, for 60 seconds on a spin-coater (e.g., available from Laurell Technologies Co., North Wales, PA, USA). Next, a glass coverslip can be placed on the PDMS mixture coated on the plastic film with one end touching the plastic film and another end elevated. In some embodiments, the stretchable surface 60 with a thickness gradient can be formed. Alternatively, the membrane can be flat. After curing at about 50 degrees Celsius (° C.), about 60° C., about 75° C., about 90° C., or about 110° C., or about 75° C. for 0.5 to 1 hour on a hotplate, the glass coverslip can be carefully removed from the stretchable surface 60 and the stretchable surface 60 can then gently peeled off from the plastic film with a razor blade. The resulting stretchable surface 60 may be trimmed using a razor blade into the desired dimensions, which in some aspects can be about 1 centimeter (cm)×about 3 cm (width×length), which can have a thickness gradient of about 300 μm on the thick end. When the stretchable surface 60 can be mounted on to the stretching device 10 between the moving component 64 and the fixed component 68, about 0.5 cm on each end can be clamped between the device 10, and hence the actual flexible PDMS membrane 60 being stretched may be about 1 cm×about 2 cm with a thickness gradient of approximately 300 micron (μm), approximately 275 μm, approximately 250 μm, approximately 220 μm, or approximately 200 μm to approximately 10 μm, approximately 30 μm, approximately 50 μm, approximately 70 μm, or approximately 100 μm, or approximately 250 μm to approximately 50 μm.

In some aspects, the stretchable surface can be treated to modify the surface for the cell cultures. In some aspects, the stretchable surface can be oxygen plasma treated at a low power setting for about 30 seconds to change the chemical property of the stretchable surface from hydrophobic to hydrophilic to facilitate cell adhesion. The resulting stretchable surface can then be coated with cell culture formulation such as rat collagen to prepare the stretchable surface for cell seeding. Cells may be seeded at a density of about 5×10⁴ cells per centimeter squared (cells/cm²) and cultured under static conditions, and then subjected to the mechanical stretch at an overall strain of between about 1.5% to about 40%, or about 15% and a frequency of about 1 Hz continuously for about 1 to about 24 hours, or about 16 hours.

The mechanical properties of the stretchable surface 60 can be determined based on the materials and geometry of the stretchable surface. As an example, to 3-D model for the gradient membrane can be first built using SolidWorks® to match the test membrane geometry. A custom material model for PDMS can then be created in the SolidWorks® simulation module. The material model can be built with the mechanical and physical properties obtained from the literature and provided in Table 1. The linear elastic isotropic material model can be used to define the linear strain behavior. The thinner side of the membrane can be fixed and a about 3 millimeter (mm) overall displacement can be set as external load on the thicker side, corresponding to an overall strain of about 15% on the membrane.

TABLE 1
Mechanical properties for PDMS

| Property | Value | Units |
|---|---|---|
| Elastic Modulus | 1.685 | MPa |
| Poisson's Ratio | 0.499 | N/A |
| Shear Modulus | 0.56 | MPa |
| Mass Density | 965 | kg/m³ |
| Tensile Strength | 5.69 | MPa |

Once formed, the strains experienced by the stretchable surface can be measured using a microscope (e.g., Evos XL Core, Thermo Fisher Scientific, Waltham, MA, USA) by examining the displacement of ink dots marked on the PDMS membrane. For the gradient membranes, three dots may be drawn on the about 1 cm×about 2 cm PDMS membrane along the stretch gradient at about 0.5 cm intervals starting from the thicker side of the membrane gradient (about 0.5 cm, about 1.0 cm, about 1.5 cm). The deformation of the dots during mechanical stretching can be recorded using the microscope, and the strain at each of the points can be quantified by analyzing the displacement of the dots using ImageJ developed at the National Institute of Mental Health, Bethesda, Maryland and available at https://imagej.net/ij/index.html.

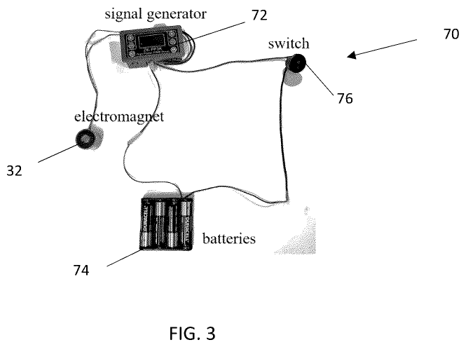

As shown in FIGS. 1A-1C, the electromagnetically driven 1-D stretching device 10 can encompass a stretching part 20 having the moving component 64, the fixed component 68, the stretchable surface 60, the track 44, the container 24, and the electromagnetic actuator 70 as shown in FIGS. 1 and 3. The moving component 64 and the fixed component 68 can be both connected to the track 44. The fixed component 68 can be connected to the base while the moving component 64 can be slid onto the track 44, with the ability to freely move with little friction acting upon it.

FIGS. 2A-2C depict the uniaxial cell stretching part 40. FIG. 2A depicts an illustration of the uniaxial cell stretching part 40, FIG. 2B depicts a cross-sectional view of the track 44 with the moving component 64, and FIG. 2C depicts the photo of the uniaxial cell stretching part 40. The stretchable surface 60 can then be secured on the device 10 between the upper and lower pieces of the fixed component 68 and the moving component 64 as about 0.5 cm of each end (both the thick and thin ends) of the stretchable surface 60 can be clamped down by conical dowels 56 (FIGS. 2A and 2C). One end of the stretchable surface 60 can be secured between the fixed component 68 while the other end can be secured between the moving component 64, allowing for uniform stretch as the moving component 64 oscillates back and forth. As shown in FIG. 2B, the moving component 64 has a trapezoidal cutout slightly larger than the track 44, which has rounded edges and can be shaped to reduce the amount of contact area to reduce friction. As such, the moving component 64 can move smoothly with a minimal amount of electromagnetic force to pull the stretchable surface 60.

FIG. 3 depicts is a top, plan, and pictorial view of an embodiment of the electromagnetic actuator 70. A rectangular piece of metal 48 can be attached onto the moving component 64 typically attracted by the electromagnet 32 and therefore can pull the moving component 64 towards the magnet with the disturbing force and be pulled back to the original position by the membrane's restoring force, creating uniform oscillations. The membrane stretch can be generated by the electromagnetic actuator 70 that can be built with an electromagnet 32 (uxcell about 5-volt (V) about 50 newton (N) Electric Lifting Magnet Electromagnet; Amazon) controlled by an electric circuit containing a pulse width modulation (PWM) signal generator 72 (ZK-PP2K pulse frequency generator; Amazon), a battery pack 74, and a switch 76 (TWTADE Rocker switch; Amazon) (FIG. 3). The PWM signal generator 72 allows the current to be turned on and off at a certain rate, and thus the frequency at which the magnet pull can be controlled. In addition, the stretching part 20 and cell culture medium can be maintained within the container 24, with the magnet holder 28 built to the outside of the container 24 (FIG. 1A). The separation of the electromagnetic actuator 70 and the cell culture medium allows for greater biocompatibility as viable cells can be maintained without exposure to contaminants. Although not wanting to be bound by theory, the magnet field strength is inversely proportional to the distance squared from the magnet 32. As a result, the magnetic field strength at the location of the metal decreases dramatically with distance. To maximize the amount of the magnetic force acting on the system, the wall between the electromagnet 32 and the metal piece 48 is as thin as possible. FIG. 3 depicts an electromagnetic actuator 70.

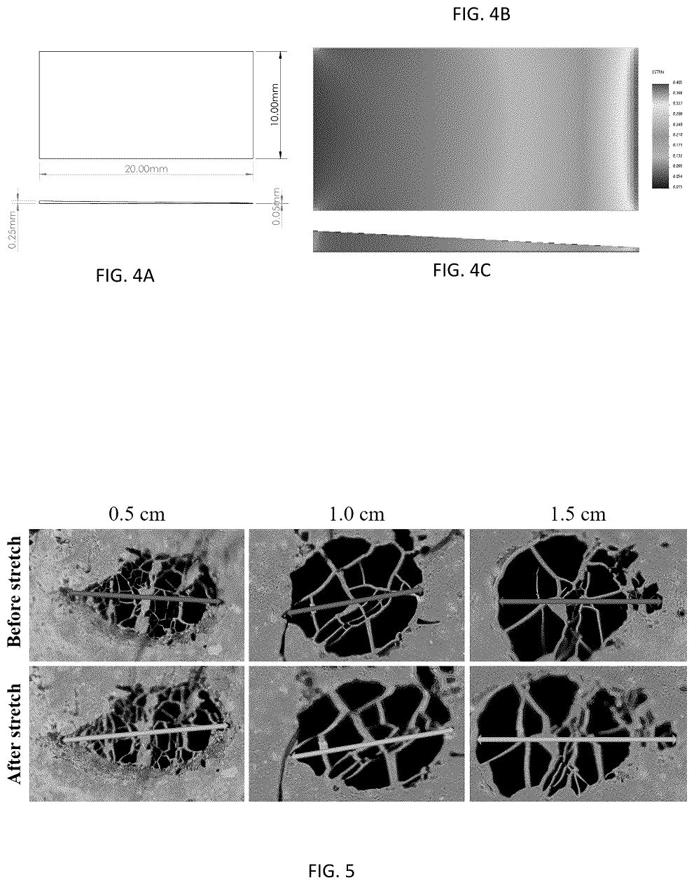

Although not wanting to be bound by theory, the stretchable surface 60 with a gradient in thickness can result in varying strains or strain rates at different locations. Along the stretch direction, the thicker areas of the membrane experienced a smaller strain than the thinner side of the membrane, which experienced greater strain. FIGS. 4A-4C depicts a simulation (as provided by SolidWorks® by Dassault Systems SolidWorks Corporation of Waltham, Massachusetts) of the strain gradient. FIG. 4A depicts the dimensions of the tapered membrane. FIG. 4B is a top view and FIG. 4C is a cross-sectional of the strain simulation. The dimensions of the membrane area stretched can be about 1 cm×about 2 cm with a thickness gradient varying from about 250 μm to about 50 μm (FIG. 4A). The material properties of polymer (e.g., PDMS) can be shown in Table 1. The simulation can be based on an overall strain of about 15%, which stretched the length from its original about 2 cm to about 2.3 cm. The SolidWorks® simulation predicted a large range of strains up to 40% (FIG. 4B). For instance, the theoretical strains at about 0.5 cm, about 1.0 cm, and about 1.5 cm from the thicker end of the PDMS membrane can be about 7.4%, about 10.0%, and about 15.8%, respectively (Table 2). Note that the strain gradient can be non-linear.

In some aspects, a ratio of the strain rate at the thicker end to a strain at the thinner end of the stretchable membrane can be at least about 1:40, at least about 1:20, at least about 1:10, at least about 1:5, at least about 1:3, or at least about 1:2, or at least about 1:1.2.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

### Cell Culture

In order to test the Human cerebral microvascular endothelial cell line (hCMEC/D3; Cat #: CLU-512, Cedarlane, Burlington, NC, USA) can be cultured in EBM-2 endothelial cell growth basal medium (Lonza, Bend, OR, USA) supplemented with EGM-2MV microvascular endothelial cell growth medium SingleQuots supplements (Lonza), about 100 units per milliliter (U/ml) penicillin, and about 100 microgram per milliliter (μg/ml) streptomycin (Life Technologies, Carlsbad, CA, USA).

The stretch device can be oxygen plasma treated at the low power setting for about 30 seconds in a plasma cleaner (Model PDC-001, Harrick Plasma, Ithaca, NY, USA) to change the chemical property of the PDMS membrane from hydrophobic to hydrophilic to facilitate cell adhesion. The PDMS membrane can then be coated with about 1 ml of 50 μg/ml rat collagen I (Corning, Corning, NY, USA) overnight in the incubator of about 37° C. and about 5% CO₂. Cells may be seeded at a density of about 5×10⁴ cells per centimeter squared (cells/cm²) and cultured under the static condition for about 4 hours, and then subjected to the mechanical stretch at an overall strain of about 15% and a frequency of about 1 Hz continuously for about 16 hours. In comparison, the cells at about 5×10⁴ cells/cm² can be cultured under the static condition for about 20 hours as the control. The cells can then be fixed for immunofluorescence staining.

### Example 2

### Immunofluorescence Staining

The cells on the membrane can be fixed with about 4% paraformaldehyde (PFA; Sigma-Aldrich, St Louis, MO, USA) for about 30 minutes at room temperature, and blocked in a phosphate buffered saline (PBS; Thermo Fisher Scientific) solution containing about 0.03 g/ml BSA, about 1% goat serum (Gibco, Grand Island, NY, USA), and about 0.2% Triton-X 100 (Sigma-Aldrich) for about 1 hour. The samples can be then incubated with Alexa Fluor 488 phalloidin (Life Technologies, about 1:about 200 in PBS solution with about 0.2% Triton-X 100) at about 4° C. overnight. The nuclei can be stained and mounted using ProLong Gold Antifade Reagent with DAPI (Life Technologies). The fluorescent images can be taken by using a Nikon Ti Eclipse Fluorescence Microscope (Nikon, Melville, NY, USA).

### Example 3

### Image Analysis

The orientation and elongation of the nuclei can be analyzed using ImageJ. The nucleus images can be extracted by adjusting threshold of color to achieve best-fitted ellipses, from which the major and minor axis can be determined. The orientation angle can be defined as the angle between the nuclear major axis and the stretch direction. The elongation can be defined by the ratio of nuclear major axis to minor axis and minus about 1.

### Example 4

### Statistical Analysis

Statistical analysis can be performed using GraphPad Prism software (GraphPad software, San Diego, CA, USA). P values between groups can be calculated using unpaired two-tailed t-test. p<about 0.05 can be considered statistically significant.

### Example 5

### Strain Determination

FIG. 5 depicts optic images of marked dots on the membrane before and after exposed to 15% overall strain. While the computational simulation displays the strain gradient, the presence of the gradient can be verified experimentally. To validate the actual strain at each of the previously mentioned points, ink dots can be drawn on the membrane at about 0.5 cm, about 1.0 cm, and about 1.5 cm from the thicker end, their deformations can be recorded, and their percentage change in length can be analyzed upon about 15% overall stretch. As shown in FIG. 5, the length of each dot along the stretch direction can be increased, and then the strain can be calculated in comparison with the original length before the stretching for each of the points. The actual strain at these locations under about 15% overall stretch can be about 9.8%, about 16.0%, and about 21.0% at about 0.5 cm, about 1.0 cm, and about 1.5 cm from the thicker end of the membrane, respectively (Table 2).

TABLE 2
Comparison of the SolidWorks ® simulation
and the measured strains of the gradient

| Location from the thicker end | 0.5 cm | 1.0 cm | 1.5 cm |
|---|---|---|---|
| Observation | 9.8% | 16.0% | 21.0% |
| Simulation gradient | 7.4% | 10.0% | 15.8% |

Table 2 compares the observed and simulated strains at different locations. Although there can be difference between these values and not wanting to be bound by theory, which likely resulted from the approximation of the dot location in the experimental characterization, these sets of data revealed the formation of the strain gradient—smaller strain at a thicker membrane while larger strain at a thinner membrane.

### Example 6

### Strain Dependent Cell Morphology

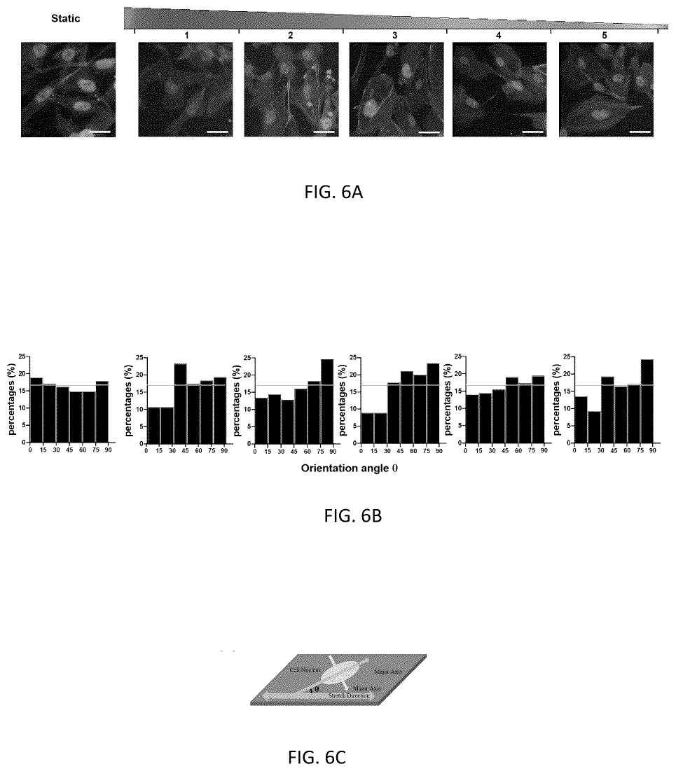

FIGS. 6A-C depict a mechanical strain-dependent cell/nuclear orientation. FIG. 6A depicts representative immunofluorescence images of hCMECs subjected to mechanical stretch of various strains in comparison with the static condition. The strain can increase from region 1 to region 5. The nuclei are stained in blue and F-actin stained in green. Scale bars are about 50 μm. FIG. 6B depicts quantitative analysis of nuclear orientation of hCMECs under the static condition and various strains. The horizontal lines represent the ideal nuclear orientation under static condition. FIG. 6C depicts an illustrative definition of the nuclear orientation angle θ.

Under the static condition, the cells spread out in all directions and displayed random cell orientation. In comparison, depending on the magnitude of the mechanical strain, the cells could orient perpendicular to the stretching direction. As shown in FIG. 6A, thicker regions of the PDMS membrane experienced smaller strains, resulting in less cell orientation in relation to the stretch direction. As the strain increased, the cells oriented themselves perpendicular to the stretch direction as seen through the nuclei and the F-actin. However, this trend diminished when the strain can be further increased at the thinner regions of the PDMS membrane. Considering the difficulty in quantifying the orientation of cells, which displayed irregular morphology, the nuclei can be more regular and easier to analyze. The orientation angle of the nucleus, as defined in FIG. 6C, reflected this strain-dependent cell orientation as well. Ideally, under the static condition, the orientation of the cells and nuclei can be completely random, and thus there should theoretically be about 16.7% of nuclear (cell) population at each of the orientation angle ranges, or each of about 6 sectors of about 15° intervals (FIG. 6B). Upon the mechanical stretching, at a smaller strain in region 1, nuclear orientation angles can be mostly in the about 30°-about 90° range with a majority of cells in the about 30°-about 45° range. As the strain increased with a decrease in the membrane thickness in the regions 2 and 3, the endothelial cells displayed a stronger nuclear orientation perpendicular to the stretch direction. For example, in region 2 there can be a greater percentage of nuclei with an orientation angle between about 45° and about 90° and most nuclei with an orientation angle between about 75° and about 90°, aligning more perpendicularly to the stretch direction. However, the trend shown in the regions 1, 2, and 3 became less significant in the regions 4 and 5. It can be speculated that the regions 4 and 5 can be overstretched, likely entering the pathogenic range of mechanical stretch (e.g., hypertension). At these regions, the cell and nuclear orientations became more randomly distributed, similar to the situation in region 1.

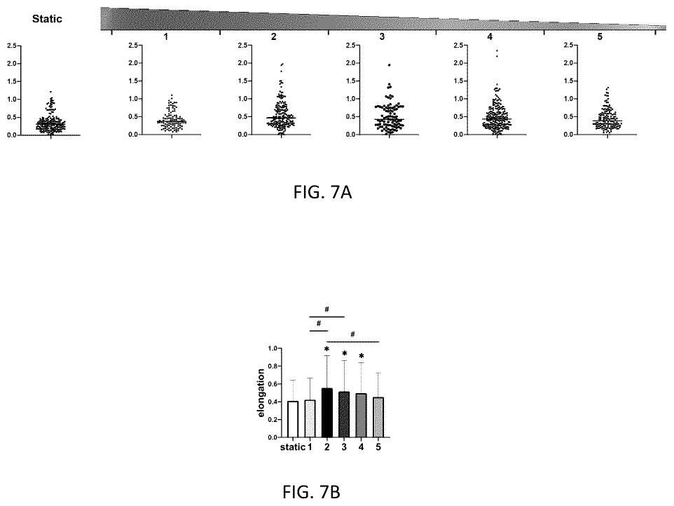

FIGS. 7A-B depict a strain-dependent nuclear elongation. FIG. 7A depicts dot plots of nuclear elongation of hCMECs under static condition and various mechanical strains. The strain increases from region 1 to region 5. FIG. 7B depicts a comparison in nuclear orientation under static condition and various strains. Static condition *: p<about 0.05 is compared to the static controls. Various strain #: p<about 0.05 is between groups.

The nuclear elongation also depicts the strain-dependent behavior. As shown in FIGS. 7A-7B, under the small strain in region 1, the nuclear elongation did not show a significant difference from the static condition. However, when the strain increased in region 2, the nuclei can be stretched more than the static condition. When the strain can be further increased from region 3 to 5, the overstretching led to deviation from the previously observed trend, resulting in the decreased nuclear elongation from region 2.

This electromagnetically driven stretching device has precise control over the strain and frequency of the mechanical stretch. In addition to the gradient of strains ranging from about 1.5% to about 40% as demonstrated in this study, uniform strains can also be generated with a PDMS membrane of uniform thickness by altering the design of the track dimension (i.e., the distance that the moving component is allowed to travel on the track). For example, when a PDMS membrane of about 200 μm in thickness can be used in the current design, a uniform strain of about 14.8±0.3% can be generated on the membrane as characterized by the aforementioned dot deformation method, which agreed with the theoretical strain value of about 15%. Furthermore, when about 5 mm displacement is used, instead of about 3 mm displacement in the current design, an overall about 25% strain can be achieved. As such, this device spans the mechanical strain from physiological (about 5-about 10%) to pathological (greater than about 20% in hypertension) range. Moreover, the PWM signal generator has a frequency range between about 1 Hz to about 150 kHz, covering the physiological frequency range. In some embodiments, a different signal generator can be used to reach a lower frequency, e.g., 0.1 Hz to cover more pathophysiological range, such 0.1 Hz to about 150 kHz. Conversely, the reported electromagnetic stretching device generated the cyclic stretch at the strain of about 1.4% and a frequency of about 0.01 Hz, not in the physiological range. Therefore, the electromagnetic stretching device can provide pathophysiologically relevant mechanical stretch. In addition, the use of the electromagnet separated from the container where the cell culture is conducted can effectively prevent the potential contamination from the stretching operation. The device can run out of battery power after several hours of cyclic stretching. To sustain the cell stretching over days even weeks, a DC power source should be adopted to run the device continuously.

When the cell study can be conducted on the strain gradient, the thinner end of the tapered PDMS membrane can occasionally break during the cyclic stretching because of the weakened mechanical strength. To resolve this problem, the membrane thickness can be increased. The SolidWorks® simulation can be conducted with a thicker, tapered PDMS membrane. The results suggest, although not wanting to be bound by theory, that a strain gradient can still be formed when increasing the overall thickness of the membrane. For example, a strain gradient of about 1.6% to about 34% can be generated on a tapered PDMS membrane with a thicker end at about 400 μm and the thinner end at about 100 μm. Although certain ranges of mechanical strains have been reported, these strain variations result from the edge effects and are heterogenous. A more controllable strain gradient has been demonstrated by applying geometrical constraints on a stretchable membrane. However, typically the strain gradient ranges between about 12% to about 18% across a distance of about 1.5 mm, and the gradient is irregular. Conversely, the strain gradients are defined and can be tailored for an end application, and additionally, at least one of a mechanical strain and a gradient can be generated over a large area, for instance, centimeter scale as demonstrated herein.

### Example 7

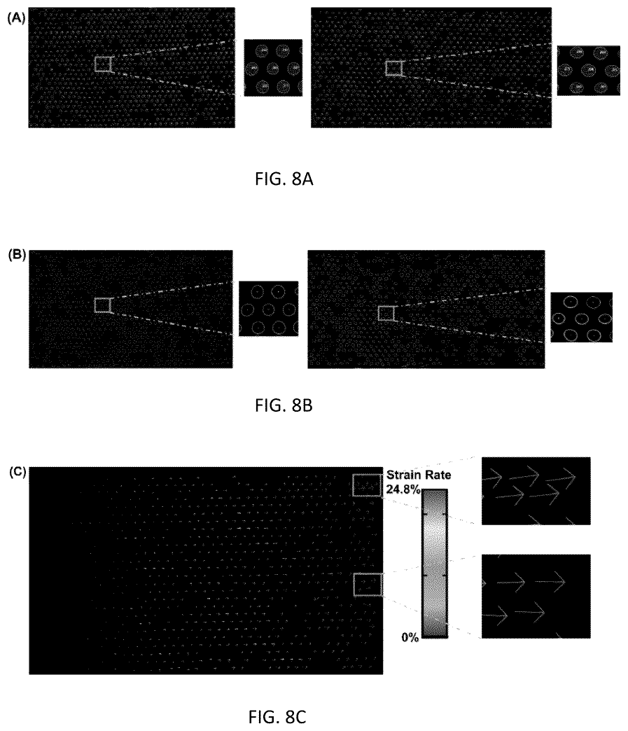

To accurately compare strain gradients, dimensions of a PDMS membrane are characterized, which confirms the presence of a strain gradient and provides the range of strain values. The strain area measures 1 cm×2 cm, and the membrane is strained to a length of 2.3 cm. A hexagonal micro pattern array is fabricated on the PDMS membrane using micro contact printing to assess the strain at each designated point. Fluorescence micrographs of both unstrained and strained membranes are acquired, with circle and ellipse recognition as depicted in FIG. 8A. The successful detection of most circles and ellipses is accomplished.

Upon removal of the original fluorescence labels (FIG. 8B), the local strain rate is determined by calculating the length ratio between the ellipse's long axis and the initial circle diameter. The strain rate and direction are visually represented in FIG. 8C through shade-coded arrows. The arrow length and shade, following a spectrum of light-to-dark, denote strain rate, while the arrowheads indicate the deformation direction.

The experimental observations are then compared with simulation results to corroborate the formation of the strain gradient. FIG. 8C demonstrates the strain rate of the PDMS membrane, revealing that the thicker direction (left) exhibits a lower strain rate, while the thinner direction shows a higher rate. Additionally, in the thinner direction, stretching along the upper and lower edges display a slight inclination (angle <7.1°) perpendicular to the stretching direction. The observed inclination can be attributed to the material's anisotropic response to the applied strain, resulting in uneven deformation across the membrane. This comprehensive interpretation of the results not only supports the understanding of the strain gradient formation, but also elucidates its implications within the defined strain area.

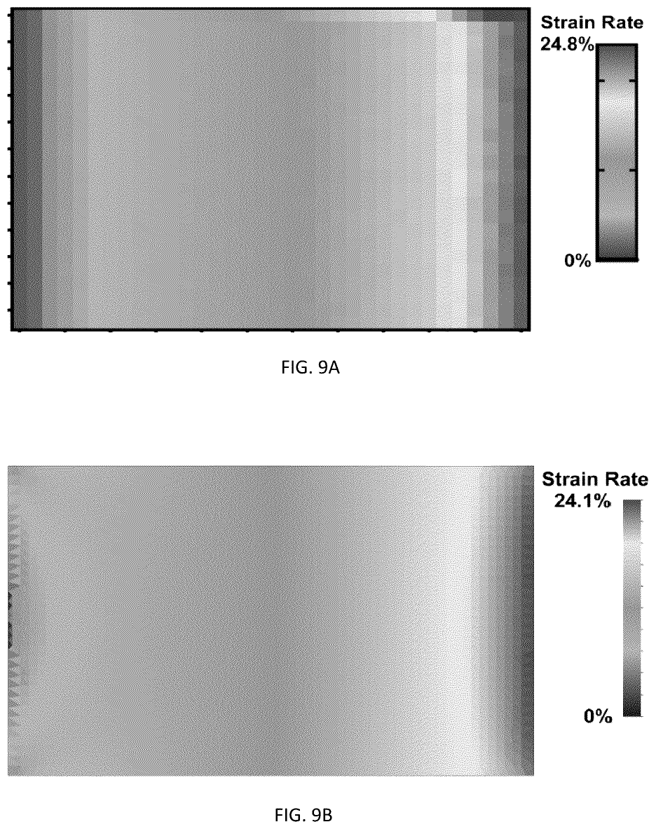

To comprehensively characterize the strain gradient, a SolidWorks® simulation is performed, with the material properties of PDMS presented in Table 1. The simulation predicts a wide range of strains from 2.9% to 24.1%, as depicted FIGS. 9A-B. The experimentally measured strain rate ranges from 2.1% to 24.8% across the membrane. The distribution of both images is highly similar, further validating the feasibility of employing simulation for strain rate estimation.

The thicker and robust membrane can allow adding nanoscale structures on the top of the membrane to replicate the nanostructured extracellular matrix (ECM) and/or put soft hydrogel on the membrane to match the ECM stiffness. In some embodiments, these nanostructures and soft hydrogel together with the pathophysiological mechanical stretch closely recapitulate the in vivo cell microenvironment.

Moreover, building this device can be inexpensive. The 3-D printing of the stretching part can incur a cost of about $2 USD and the printing charge can be about $30 USD. The electromagnet, function signal generator, and the switch can be $11 USD, $16 USD, and $1 USD, respectively. These costs, together with other small parts such as wires, batteries, and PDMS membrane, can be less than about $70 USD in comparison with several thousand dollars for a commercial Flexcell Systems. To sum up, in some embodiments, the electromagnetic stretching device is simple, versatile, and cost-effective, suitable for the study of mechanical stretch effects.

Furthermore, the cell study exhibited that the microvascular endothelial cells responded differently to the alteration in mechanical strain. Physiologically relevant mechanical strain can effectively orient the endothelial cells perpendicular to the stretch direction while excessive strain may harm the endothelial phenotype. These observations agree with the in vivo results. The functionality of tight junctions between adjacent endothelial cells is significant because they form an adjacent barrier between cells to prevent the leakage of molecules and ions through the plasma membranes of adjacent cells. Tight junctions can become impaired under pathological stress, leading to increased membrane permeability, or leaky endothelium. Pathophysiological mechanical stretching may affect the formation and function of vascular endothelium.

Having described various systems and methods herein, certain embodiments can include, but are not limited to:

In an aspect, an electromagnetically driven one-dimensional stretching device, includes a stretching part and an electromagnetic actuator.

A second aspect can include the electromagnetically driven one-dimensional stretching device of the first aspect, wherein the stretching part comprises a track, one or more clamps, a container, and optionally one or more dowels.

A third aspect can include the electromagnetically driven one-dimensional stretching device of the first aspect or the second aspect, wherein the stretching part comprises a polydimethylsiloxane membrane.

A fourth aspect can include the electromagnetically driven one-dimensional stretching device of any one of the proceeding aspects, wherein the stretching part comprises a fixed component and a moving component.

A fifth aspect can include the electromagnetically driven one-dimensional stretching device of any one of the proceeding aspects, wherein the polydimethylsiloxane membrane comprises dimensions of about 1 cm×about 2 cm with a thickness gradient of about 250 μm to about 50 μm.

A sixth aspect can include a method of forming an electromagnetically driven one-dimensional stretching device of any one of the proceeding aspects, including spin-coating a weight ratio of about 10:about 1.05 of a polydimethylsiloxane resin: a curing agent mixture.

A seventh aspect can include the method of forming an electromagnetically driven one-dimensional stretching device of any one of the proceeding aspects, further including the spin-coating the polydimethylsiloxane resin and the curing agent mixture on an overhead transparency plastic film.

An eighth aspect can include the method of forming an electromagnetically driven one-dimensional stretching device of any one of the proceeding aspects, wherein the spin-coating is at about 300 revolutions-per-minute for 60 seconds.

A ninth aspect can include the method of forming an electromagnetically driven one-dimensional stretching device of any one of the proceeding aspects, further comprising the spin-coating the polydimethylsiloxane resin and the curing agent mixture on one end touching an overhead transparency plastic film and another end by two substantially identical glass coverslips to form a thickness gradient.

A tenth aspect can include the method of forming an electromagnetically driven one-dimensional stretching device of any one of the proceeding aspects, further comprising curing at about 75° C. for about 1 hour to form a polydimethylsiloxane membrane.

An eleventh aspect can include the method of forming an electromagnetically driven one-dimensional stretching device of any one of the proceeding aspects, further comprising mounting the polydimethylsiloxane membrane on the stretching part.

A twelfth aspect can include the method of forming an electromagnetically driven one-dimensional stretching device of any one of the proceeding aspects, further comprising mounting the polydimethylsiloxane membrane to the stretching part between a fixed component and a moving component.

A thirteenth aspect can include the method of forming an electromagnetically driven one-dimensional stretching device of any one of the proceeding aspects, further comprising clamping each end of the polydimethylsiloxane membrane on the stretching part.

A fourteenth aspect can include the method of forming an electromagnetically driven one-dimensional stretching device of any one of the proceeding aspects, wherein the polydimethylsiloxane membrane comprises dimensions of about 1 cm×about 2 cm with a thickness gradient of about 250 μm to about 50 μm.

A fifteenth aspect can include the method of forming an electromagnetically driven one-dimensional stretching device of any one of the proceeding aspects, including spin-coating a weight ratio of about 10:about 1.05 of a polydimethylsiloxane resin: a curing agent mixture on a plastic film wherein the polydimethylsiloxane resin and the curing agent mixture are spin-coated on one end touching the plastic film and another end by two substantially identical glass members to form a thickness gradient.

A sixteenth aspect can include the method of forming an electromagnetically driven one-dimensional stretching device of any one of the proceeding aspects, wherein the plastic film comprises an overhead transparency plastic film and the two substantially identical glass members comprise, independently, glass coverslips.

In a seventeenth aspect, a method of making an electromagnetically driven one-dimensional stretching device, includes spin-coating a weight ratio of about 20:1 to about 1:1 of a polydimethylsiloxane resin: a curing agent mixture to a polydimethylsiloxane membrane.

An eighteenth aspect can include the method of the seventeenth aspect, wherein the weight ratio is about 10:about 1.05 of the polydimethylsiloxane resin: the curing agent mixture to the polydimethylsiloxane membrane.

For purposes of the disclosure herein, the term “comprising” includes “consisting” or “consisting essentially of” Further, for purposes of the disclosure herein, the term “including” includes “comprising,” “consisting,” or “consisting essentially of.”

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

While embodiments of the invention have been shown and described, modifications thereof can be made by one skilled in the art without departing from the spirit and teachings of the invention. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L}, and an upper limit, R_{U}, is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, i.e., k is 1 percent, 2 percent, 3 percent, 4 percent, 5 percent, 50 percent, 51 percent, 52 percent, 95 percent, 96 percent, 97 percent, 98 percent, 99 percent, or 100 percent. Moreover, any numerical range defined by two R numbers as defined above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

What is claimed is:

1. An electromagnetically driven one-dimensional stretching device, comprises:
a stretchable surface; and
an electromagnetic actuator coupled to the stretchable surface, wherein the electromagnetic actuator is configured to stretch the stretchable surface along one-dimension.

2. The electromagnetically driven one-dimensional stretching device of claim 1, further comprising a stretching part configured to hold the stretchable surface, wherein the stretching part comprises a track, one or more clamps, a container, and one or more dowels.

3. The electromagnetically driven one-dimensional stretching device of claim 1, wherein the stretchable surface comprises a polydimethylsiloxane membrane.

4. The electromagnetically driven one-dimensional stretching device of claim 1, wherein the stretching part comprises a fixed component and a moving component.

5. The electromagnetically driven one-dimensional stretching device of claim 3, wherein the polydimethylsiloxane membrane comprises dimensions of about 1 cm×about 2 cm with a thickness gradient of about 250 μm to about 50 μm.

6. A method of forming an electromagnetically driven one-dimensional stretching, the method comprising:
spin-coating a weight ratio of about 10:1 about 1.05:1 of a polydimethylsiloxane resin to a curing agent mixture on a surface to form a stretchable surface;
removing the stretchable surface from the surface;
coupling an electromagnetic actuator to the stretchable surface; and
stretching the stretchable surface along one-dimension using the electromagnetic actuator.

7. The method of claim 6, further comprising:
spin-coating the polydimethylsiloxane resin and the curing agent mixture on a plastic film.

8. The method of claim 6, wherein the spin-coating occurs at about 300 revolutions-per-minute for 60 seconds.

9. The method of claim 6, further comprising:
spin-coating the polydimethylsiloxane resin and the curing agent mixture on one end touching a plastic film and another end by two glass coverslips to form a thickness gradient.

10. The method of claim 6, further comprising:
curing the polydimethylsiloxane resin at about 75° C. for about 1 hour to form the stretchable surface.

11. The method of claim 6, further comprising mounting the stretchable surface on a stretching part.

12. The method of claim 11, further comprising:
mounting the stretchable surface to the stretching part between a fixed component and a moving component.

13. The method of claim 11, further comprising:
clamping each end of the stretchable surface on the stretching part.

14. The method of claim 11, wherein the stretchable surface comprises dimensions of about 1 cm×about 2 cm with a thickness gradient of about 250 μm to about 50 μm.

15. The method of claim 11, wherein the stretching part comprises a track, one or more clamps, a container, and one or more dowels.

16. The method of claim 11, wherein the stretching part is 3-D printed.

17. A method of forming strain gradient stretchable surface, the method comprising:
spin-coating a weight ratio of about 20:1 to about 1:1 of a polydimethylsiloxane resin to a curing agent mixture on a plastic film, wherein the polydimethylsiloxane resin and the curing agent mixture are spin-coated on one end touching the plastic film and another end by two glass members to form a thickness gradient.

18. The method of claim 17, wherein the plastic film comprises a transparency plastic film and the two substantially identical glass members comprise, independently, glass coverslips.

19. A method of making an electromagnetically driven one-dimensional stretching device, comprising spin-coating a weight ratio of about 15:1 to about 1:1 of a polydimethylsiloxane resin: a curing agent mixture to a polydimethylsiloxane membrane.

20. The method of claim 17, wherein the weight ratio is about 10:1 to about 1.05:1 of the polydimethylsiloxane resin: the curing agent mixture to the polydimethylsiloxane membrane.
